(19) United States
(12) Patent Application Publication
(10) Pub. No.: US 2025/0174046 A1
(43) Pub. Date: May 29, 2025

(54) METHOD AND SYSTEM FOR CALCULATING CALORIES BURNED DURING ACTIVITY PERFORMANCE USING ARTIFICIAL INTELLIGENCE (AI)

(71) Applicant: RAJIV TREHAN, BANGKOK (TH)

(72) Inventor: RAJIV TREHAN, BANGKOK (TH)

(73) Assignee: RAJIV TREHAN, (omitted)

(21) Appl. No.: 18/958,278

(22) Filed: Nov. 25, 2024

Related U.S. Application Data
(60) Provisional application No. 63/602,472, filed on Nov. 24, 2023.

(51) Int. Cl. A61B 5/00; A63B 24/00

(52) U.S. Cl. CPC G06V 40/23; A63B 24/0062; G06V 10/70; G16H 20/30; A63B 2230/75

(57) ABSTRACT

A method for calculating calories burned during activity performance using Artificial Intelligence (AI) is disclosed. The method includes receiving video stream of user performing activity and set of user profile attributes of the user. The method further includes creating, for each of the plurality of frames, multimedia vector corresponding to the associated frame that are further processed to determine set of activity parameters associated with user. The method includes selecting a target user from plurality of target users based on similarity between set of user profile attributes of the user and set of target profile attributes of the target user. The method further includes comparing each of the set of activity parameters with corresponding activity parameter from a set of target activity parameters associated with the target user. The method includes determining user efficiency level and count of calories burned by the user based on the result of comparing.

### TECHNICAL FIELD

This disclosure relates generally to calories determination during activity performance, and more particularly to a method and system for calculating calories burned during activity performance using Artificial Intelligence (AI).

In the era of modernization and urbanization, people nowadays have become more aware and concerned about maintaining physical and mental fitness. One of the best ways of maintaining physical fitness is regular workout sessions and monitoring overall calories burnt during a given workout session. In order to determine calories burned by a user or a current calorie burn rate of the user, the user may use various wearables or applications that are currently available in the market. Examples of these wearables may include, but are not limited to smart bands, smart watches, smart rings, or other similar smart devices that can be worn on a specific body part of the user. These wearables use an ensemble of sensors, and statistical or historic information to estimate total calories burnt by a user while performing a given activity. Examples of sensors may include pedometers, heart rate monitors, pulse oximeters, accelerometers, gyroscopes, and temperature sensors.

Existing wearables or applications have a lot of limitations. These limitations, for example, may include non-reliance on individual variability, inaccurate baseline data (e.g., inaccurate weight, height, age, etc.), limited activity recognition, non-reliance on environmental factors (e.g., weather, terrain, temperature, etc.), non-reliance on heart rate variability, inaccurate caloric equations, no metabolic adaptation, or bad quality of data input. Moreover, existing applications and wearables neither specify an individual's accuracy in performing a given activity nor provide real-time feedback to the individual, especially with respect to calorie burn rate.

One of the reasons is complete reliance on sensors to capture data by sensing movement of the individual. Another one of the reasons may be to conserve battery life of a wearable by avoiding the need to provide real-time feedback. These sensors do not precisely capture real-time parameters associated with the activity (for example, accuracy of performing the exercise, degree of freedom of the activity being performed, periodicity of the activity, and the like) being performed by the individual. Also, existing wearables or applications are not able to accurately identify the current activity being performed by a given individual. The individual in most of the cases may have to manually provide an input to the wearable or the application that a specific type of activity is being performed by the individual.

These existing applications and wearables generally rely on agreed information on the calories burned by the individual performing a given activity for a defined period. The calculations of calories using these applications and wearables generally include parameters such as the weight of the individual (i.e., an exerciser) or their Body Mass Index (BMI). The calculations also assume that the exerciser is performing the exercise at an expected pace, i.e., an expected number of repetitions being performed within the time period. However, none of these existing applications and wearables check how vigorously and at what intensity the individual is performing the exercise.

Further, none of these applications and wearables is able to accurately distinguish different exercises with varying energy expenditure, which in turn affects the calorie estimations made for these exercises. Due to these reasons, the heart rate and time-based calorie estimations made by the existing applications and wearables are often inaccurate. Currently, to precisely determine the calories being burned by the individual during the activities requires the use of high-tech medical equipment. Further, there may be scenarios where people may not have wearable devices or may not be interested in wearing any additional devices for estimating calories. In these scenarios, the calories estimated using the applications are not precise due to the above listed reasons.

### SUMMARY

In one embodiment, a method for calculating calories burned during activity performance using Artificial Intelligence (AI) is disclosed. In one example, the method may include receiving, in real-time, a video stream of a user performing an activity and a set of user profile attributes of the user. It should be noted that the video stream may include a plurality of frames. The method may further include creating, for each of the plurality of frames, a multimedia vector corresponding to the associated frame. The method may further include processing, by an AI model, the multimedia vector created for each of the plurality of frames to determine a set of activity parameters associated with the user. The method may further include selecting, by the AI model, a target user from a plurality of target users based on similarity between the set of user profile attributes of the user and a set of target profile attributes of the target user. It should be noted that the target user corresponds to a benchmark user. The method may further include identifying a set of target activity parameters associated with the target user. The method may further include comparing, by the AI model, each of the set of activity parameters with a corresponding activity parameter from the set of target activity parameters. The method may further include determining, in real-time, contemporaneous to the user performing the activity, a user efficiency level based on a result of the comparing. The method may further include calculating, by the AI model, in real-time, contemporaneous to the user performing the activity, a count of calories burned by the user based on the user efficiency level.

In another embodiment, a system for calculating calories burned during activity performance using AI is disclosed. In one example, the system may include a processor and a computer-readable medium communicatively coupled to the processor. The computer-readable medium may store processor-executable instructions, which, on execution, may cause the processor to receive, in real-time, a video stream of a user performing an activity and a set of user profile attributes of the user. It should be noted that the video stream may include a plurality of frames. The processor-executable instructions, on execution, may further cause the processor to create, for each of the plurality of frames, a multimedia vector corresponding to the associated frame. The processor-executable instructions, on execution, may further cause the processor to process, by an AI model, the multimedia vector created for each of the plurality of frames to determine a set of activity parameters associated with the user. The processor-executable instructions, on execution, may further cause the processor to select, by the AI model, a target user from a plurality of target users based on similarity between the set of user profile attributes of the user and a set of target profile attributes of the target user. It should be noted that the target user corresponds to a benchmark user. The processor-executable instructions, on execution, may further cause the processor to identify a set of target activity parameters associated with the target user. The processor-executable instructions, on execution, may further cause the processor to compare, by the AI model, each of the set of activity parameters with a corresponding activity parameter from the set of target activity parameters. The processor-executable instructions, on execution, may further cause the processor to determine, in real-time, contemporaneous to the user performing the activity, a user efficiency level based on a result of the comparing. The processor-executable instructions, on execution, may further cause the processor to calculate, by the AI model, in real-time, contemporaneous to the user performing the activity, a count of calories burned by the user based on the user efficiency level.

In yet another embodiment, a method for calculating calories burned during activity performance using AI is disclosed. In one example, the method may include receiving, in real-time, a video stream of a user performing an activity and a set of user profile attributes of the user. It should be noted that the video stream may include a plurality of frames. The method may further include creating, for each of the plurality of frames, a multimedia vector corresponding to the associated frame. The method may further include processing, by an AI model, the multimedia vector created for each of the plurality of frames to determine a set of activity parameters associated with the user. The method may further include comparing, by the AI model, each of the set of activity parameters with a corresponding activity parameter from a set of target activity parameters. It should be noted that the set of target activity parameters corresponds to the activity being performed by a benchmark user. The method may further include determining, in real-time, contemporaneous to the user performing the activity, a user efficiency level based on a result of the comparing. The method may further include adapting, by the AI model, the user efficiency level based on the set of user profile attributes of the user and a set of benchmark profile attributes of the benchmark user. The method may further include calculating, by the AI model, in real-time, contemporaneous to the user performing the activity, a count of calories burned by the user based on the adapted user efficiency level.

In yet another embodiment, a system for calculating calories burned during activity performance using AI is disclosed. In one example, the system may include a processor and a computer-readable medium communicatively coupled to the processor. The computer-readable medium may store processor-executable instructions, which, on execution, may cause the processor to receive, in real-time, a video stream of a user performing an activity and a set of user profile attributes of the user. It should be noted that the video stream may include a plurality of frames. The processor-executable instructions, on execution, may further cause the processor to create, for each of the plurality of frames, a multimedia vector corresponding to the associated frame. The processor-executable instructions, on execution, may further cause the processor to process, by an AI model, the multimedia vector created for each of the plurality of frames to determine a set of activity parameters associated with the user. The processor-executable instructions, on execution, may further cause the processor to compare, by the AI model, each of the set of activity parameters with a corresponding activity parameter from a set of target activity parameters. It should be noted that the set of target activity parameters corresponds to the activity being performed by a benchmark user. The processor-executable instructions, on execution, may further cause the processor to determine, in real-time, contemporaneous to the user performing the activity, a user efficiency level based on a result of the comparing. The processor-executable instructions, on execution, may further cause the processor to adapt, by the AI model, the user efficiency level based on the set of user profile attributes of the user and a set of benchmark profile attributes of the benchmark user. The processor-executable instructions, on execution, may further cause the processor to calculate, by the AI model, in real-time, contemporaneous to the user performing the activity, a count of calories burned by the user based on the adapted user efficiency level.

It is to be understood that both the foregoing general description and the following detailed description are exemplary and explanatory only and are not restrictive of the invention, as claimed.

### BRIEF DESCRIPTION OF THE DRAWINGS

The accompanying drawings, which are incorporated in and constitute a part of this disclosure, illustrate exemplary embodiments and, together with the description, serve to explain the disclosed principles.

FIG. 1 illustrates a functional block diagram of a computing device that uses Artificial Intelligence (AI) to accurately calculate calories burned by a user performing an activity, in accordance with some embodiments.

FIG. 2 illustrates an exemplary scenario where profile attributes of a user are being captured and subsequently displayed via a Graphical User Interface (GUI), in accordance with some embodiments.

FIG. 3 illustrates an exemplary GUI displaying user selection of an activity from a plurality of activities, in accordance with some embodiments.

FIG. 4 illustrates an exemplary GUI displaying selection of an activity and corresponding activity parameters, in accordance with some embodiments.

FIG. 5 illustrates an exemplary scenario of a user performing an activity selected from a user device and a GUI displaying a current set of activity parameters of the user, in accordance with some embodiments.

FIG. 6 illustrates an exemplary scenario of selection of a target user matching with a current user from amongst a plurality of target users, in accordance with some embodiments.

FIG. 7 illustrates an exemplary GUI displaying performance details of a user performing a selected activity, in accordance with some embodiments.

FIG. 8 illustrates a flowchart of an exemplary process for calculating calories burned during activity performance using AI, in accordance with some embodiments.

FIG. 9 illustrates a flowchart of an exemplary process for selecting a target user from a plurality of target user, in accordance with some embodiments.

FIG. 10 illustrates a flowchart of an exemplary process for identifying a set of target activity parameters, in accordance with some embodiments.

FIG. 11 illustrates a flowchart of another exemplary process for calculating calories burned during activity performance using AI, in accordance with some embodiments.

FIG. 12 is a block diagram of an exemplary computer system for implementing embodiments consistent with the present disclosure.

### DETAILED DESCRIPTION

Exemplary embodiments are described with reference to the accompanying drawings. Wherever convenient, the same reference numbers are used throughout the drawings to refer to the same or like parts. While examples and features of disclosed principles are described herein, modifications, adaptations, and other implementations are possible without departing from the spirit and scope of the disclosed embodiments. It is intended that the following detailed description be considered as exemplary only, with the true scope and spirit being indicated by the following claims.

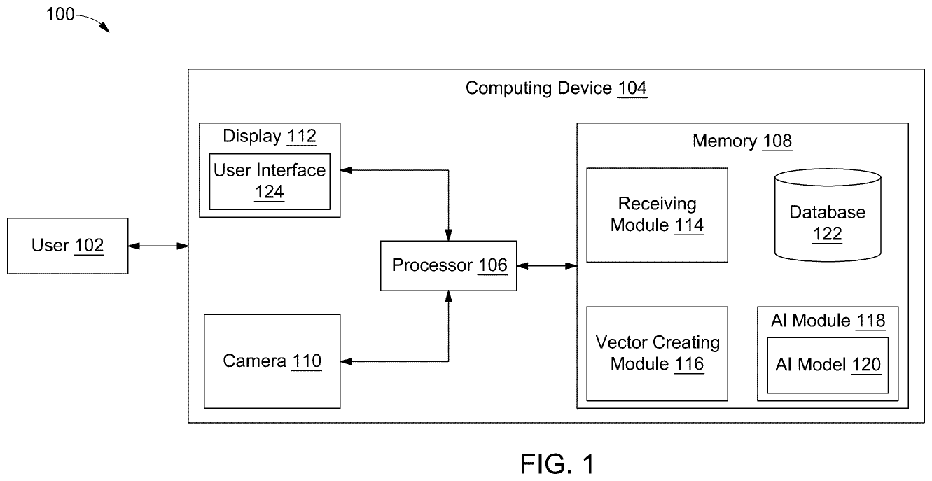

Referring now to FIG. 1, a functional block diagram 100 of a computing device 104 that uses Artificial Intelligence (AI) to accurately calculate calories burned by a user 102 performing an activity is illustrated, in accordance with some embodiments. The computing device 104, for example, may be a server, a laptop, a tablet, a notebook, a netbook, a smartphone, a mobile phone, an application server, or any other computing device. The computing device 104 may be configured to use AI in order to calculate total number of calories burned by the user 102 while performing an activity. Examples of the activity may include, but are not limited to an aerobic activity, a cardiovascular activity, a strength training activity, physiotherapy, yoga, boxing, guided meditations, physiotherapy, flower arranging, origami, dance, theatre, any form of performing arts, martial arts, speech therapy, rehab, drawing, painting, physical therapy and rehabilitation, CrossFit, Les Mills, F45, Zumba, Bikram Yoga, or Orange Theory.

The computing device 104 may include one or more processors 106, a memory 108, a camera 110, and a display 112. The memory 108 may be a non-volatile memory (e.g., flash memory, Read Only Memory (ROM), Programmable ROM (PROM), Erasable PROM (EPROM), Electrically EPROM (EEPROM) memory, etc.) or a volatile memory (e.g., Dynamic Random Access Memory (DRAM), Static Random-Access memory (SRAM), etc.) The memory 108 may include a receiving module 114, a vector creating module 116, an AI module 118, and a database 122. Further, the AI module 118 may include an AI model 120. Examples of the AI model 120 may include, but are not limited to a Linear regression model, a Logistic regression model, a Linear discriminant analysis model, a Naive Bayes model, a K-Nearest Neighbors model, a support vector machine model, a deep neural network model, a random forest model, and the like. In some embodiments, the AI model 120 may be a fined-tuned generative AI model. The memory 108 may store instructions that, when executed by the one or more processors 106, may cause the one or more processors 106 to calculate, using AI, the calories burned by the user 102 while performing an activity, in accordance with aspects of the present disclosure.

Initially, the receiving module 114 may receive a video or video-stream of the user 102 performing an activity and a set of user profile attributes of the user 102. The video may be a real-time, near real-time video, or a pre-recorded video. By way of an example, the set of user profile attributes may include, but may not be limited to a body type, age, gender, weight, height, Body Mass Index (BMI), and physical abilities (such as balance, agility, stamina, endurance, coordination, speed, and the like while performing the activity). Body type, for example, may be an endomorph, a mesomorph, or an ectomorph. The user 102 may be a person who is interested in accurate, timely, and real-time computation of calories burned by them while performing the activity. It may be noted that in sharp contrast to the current disclosure, the wearables have the drawback of providing inaccurate and asynchronous computation of calories burned by a user. The current disclosure aims to overcome these drawbacks by way of the invention.

The video stream of the user 102 while performing the activity may be captured through a camera 110. It may be noted that the video may include a plurality of frames. In some embodiment, the set of user profile attributes may automatically be determined by the computing device 104. To this end, the computing device 104 may initially instruct the user, via a user interface 124 to stand in front of the camera 110 facing forward and then facing backwards, the camera 110 may then capture (or scan) a full body image of the user 102. Once the image of the user 102 is scanned, the AI module 118 may process, via the AI model 120 using computer vision technology, the full body image of the user 102 to determine the set of user profile attributes. In some embodiments, the set of user profile attributes of the user 102 may be received as an input from the user 102 via the user interface 124. In some other embodiments, the user profile attributes of the user 102 may be extracted from a third-party database using a common user identifier (for example, cell phone of the user 102). In some embodiments, the video of the user 102 while performing the activity may be captured through a camera that is external to the computing device 104. Further, the video may be uploaded to the computing device 104 via the user 102.

Further, once the video and the set of profile attributes of the user 102 is received, the vector creating module 116 may create, for each of the plurality of frames, a multimedia vector corresponding to the associated frame. Upon creating the multimedia vectors, the vector creating module 116 may send the multimedia vectors to the AI module 118. Further, the AI module 118 may process, via the AI model 120, the multimedia vector created for each of the plurality of frames to determine a set of activity parameters associated with the user 102. By way of an example, the set of activity parameters may include, but may not be limited to a type of activity, a pace of performing the activity, number of repetitions of the activity, periodicity (i.e. rhythm) of the activity, duration of performing the activity, an accuracy (i.e. correct posture) of performing the activity, range of motion, and a degree of freedom of the activity. It may be noted that the AI model 120 using computer vision technology may automatically identify the activity that is being performed by the user 102. Thus, without requiring any input from the user 102, the activity being performed by the user 102 may be identified. Identification of the activity is pivotal to arrive at an accurate estimate of calories burned by the user 102 while and after performing the activity.

Once the set of activity parameters of the user 102 is determined, the AI module 118 may select, via the AI model 120, a target user from a plurality of target users based on similarity between the set of user profile attributes of the user 102 and a set of target profile attributes of the target user. By way of an example, the set of target profile attributes may include, but may not be limited to a body type, age, gender, weight, height, and other physical abilities (such as balance, coordination, speed while doing the activity). The target user may correspond to a benchmark user who has similar or nearly similar profile attributes as the user 102. Additionally, a benchmark user is a user whose profile attributes and activity parameters for the specific activity (that user 102 is currently performing) may be available in a database 122 or any other database available on the cloud (not shown in FIG. 1). Further, as will be appreciated, the benchmark user may be any person who has been clinically and/or accurately monitored for calorie expenditure estimation for the specific activity being performed by the user 102.

In order to identify the target user from the plurality of user, the AI module 118, via the AI model 120, may determine similarity of the set of user profile attributes with the corresponding set of target profile attributes of each of the plurality of target users. Further, the AI module 118 may calculate, via the AI model 120, a similarity score between each of the set of user profile attributes and each of the corresponding set of target profile attributes. Byway of an example, the similarity score may be calculated between each of the set of user profile attributes and each of the corresponding set of target profile attributes using a similarity analysis. The similarity analysis may be performed by the AI model 120. Alternatively, the similarity analysis may be performed using one of Euclidean distance, Manhattan distance, Minkowski distance, and Chebyshev distance. The similarity score range may fall between 0-1 or any other range decided by an administrator. Thereafter, the AI module 118 may select, via the AI model 120, the target user based on the calculated similarity score. It should be noted that the similarity score calculated for the target user is the highest.

Once the target user is selected, the AI module 118 may identify, via the AI model 120, a set of target activity parameters associated with the target user. It may be apparent that the set of target activity parameters may have been computed beforehand for multiple target users. It may further be noted that in order to determine the set of target activity parameters, the receiving module 114 may first receive a video of the target user performing the target activity and the set of target profile attributes of the target user. The vector creating module 116 may create a multimedia vector corresponding to each of a plurality of frames in the video of the target user. Once the multimedia vector is created, the AI module 118 may process, via the AI model 120, the multimedia vector created for each of the plurality of frames to determine the set of target activity parameters associated with the target user. The AI module 118 may store the set of target activity parameters associated with the target user in the database 122. Thus, when the target user is selected, the AI module 118 may retrieve the set of target activity parameters associated with the target user from the database 122. The set of target activity parameters may include, for example, a type of activity, a pace of performing the activity, number of repetitions of the activity, an accuracy of performing the activity, range of motion, a degree of freedom of the activity being performed, periodicity (i.e., rhythm) of the activity, duration for which the activity was performed, and the like.

Once the set of target activity parameters is determined, the AI module 118 may determine, via the AI model 120, an estimated target calories for the target activity based on a count of calories burned by the target user corresponding to the target activity. Further, the AI model 120 may compute a target efficiency level corresponding to the target activity based on the estimated target calories. The AI model 120 may store the target estimated calories and the target efficiency level in the database 122. It should be noted that the target efficiency level of the target user for the target activity may be 100%.

Once the set of activity parameters and the set of target activity parameters is determined, the AI module 118 may compare, via the AI model 120, each of the set of activity parameters with a corresponding activity parameter from the set of target activity parameters in real-time contemporaneous to the user 102 performing the activity. Byway of an example, the pace of the activity being performed by the user 102 may be compared with the pace of the activity that was performed by the target user. Similarly, the number of repetitions of the activity being performed by the user 102 may be compared with the number of repetitions that were performed by the target user. In a similar manner, each of the set of activity parameters of the user 102 may be compared with a corresponding activity parameter from the set of target activity parameters.

Further, upon comparison, the AI module 118 may determine, via the AI model 120, a user efficiency level based on a result of the comparing. A result of comparing, for example, may be variance in specific activity parameters of the user 102 versus that of the target user. By way of an example, if the number of repetitions for a given activity within a given time period for the target user was 20, while that of the user 102 is 10, then the variance is that of 50%. In other words, efficiency level of the user 102 for this specific activity parameter is 50%. In a similar manner, such variance may be determined for each activity parameter and the user efficiency level may then be arrived at based on a function (for example, weighted average) of such variance. It will be apparent that the above example is used for ease of explanation and more complex techniques may be used to determine efficiency level of the user 102 per activity parameter. Complex techniques may involve use of algorithmic, statistical, or AI based solutions.

In some embodiments, the user efficiency level may further be varied by the AI model 120 based on some external factors, for example, the environment where the user 102 may be performing the activity. The environment may include temperature, air quality, humidity, distraction, noise level, time, day, or the like. By way of an example, the day of the week may indeed influence activity efficiency level due to variations in lifestyle, routine, and mental energy across different days. Such external factors may further have an effect on the efficiency level of the user 102. Accordingly, an adjustment may be made to the determined user efficiency level. For example, in case the user 102 is working in a humid environment, user efficiency may dip. As a result, the determined user efficiency level may be adjusted with a slight increment to compensate for the humid environment.

In some embodiments, the AI module 118 may not be able to find (or select) a target user who has same or similar set of user profile attributes from the database 122. In other words, a target user who has similar or same profile attributes as the user 102 is not currently available in the database 122. In such a case, the AI module 118 may compare each of the set of activity parameters with a corresponding activity parameter from the set of target activity parameters associated with a benchmark user. A benchmark user is a user whose profile attributes and activity parameters for the specific activity that the user 102 is currently performing may be available in the database 122. Thus, in this case, the set of target activity parameters may correspond to the activity being performed by a benchmark user. As will be appreciated, the benchmark user may be any person who has been clinically and/or accurately monitored for calorie expenditure for the target activity.

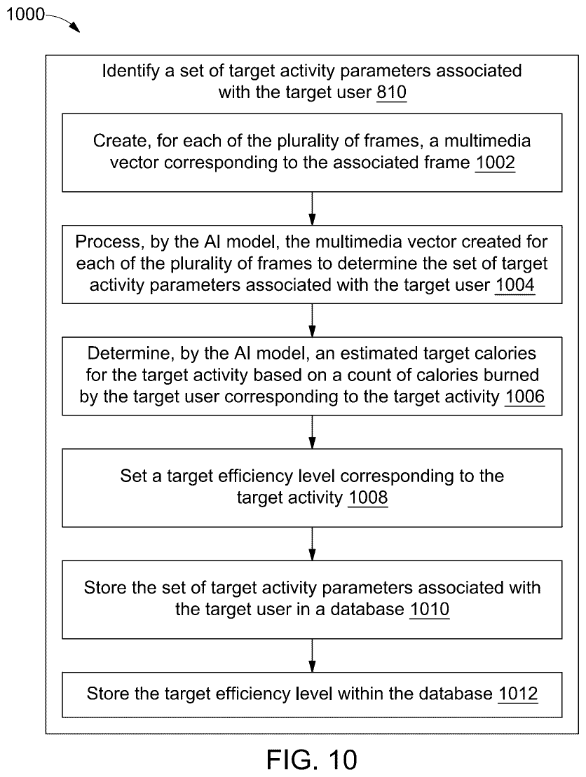

The AI module 118 may then determine, via the AI model 120, the user efficiency level based on a result of the comparing. Once the user efficiency level is determined, the AI module 118 may adapt, via the AI model 120, the user efficiency level based on the set of user profile attributes of the user and a set of benchmark profile attributes of the benchmark user. The AI module 118, via the AI model 120, may determine a deviation of one or more of the set of user profile attributes relative to the associated attributes from the set of benchmark profile attributes. Based on the determined deviation, the AI module 118 may adjust the user efficiency level. This is further explained in greater detail in conjunction with FIG. 10.

Once the user efficiency level is determined, the AI module 118 may calculate, via the AI model 120, a count of calories burned by the user 102 based on the user efficiency level. By way of an example, if the user efficiency level is determined as 50% and the count of calories burned by the target user (or the benchmark user) was 300 calories, then the total count of calories burnt by the user 102 would be 150 calories. It will be apparent that the current example is used for ease of explanation, however, a linear mapping as explained in the above example may not be actually implemented. Adjustments in the efficiency of the user 102 may be performed statistically, Algorithmically, or by using AI. By way of an example, 50% less efficiency of the user 102 may actually translate to a 20% difference in the calorie burn rate or the total calories burned by the user 102.

Further, the AI module 118 may render, via the user interface 124 (Graphical User Interface (GUI)) on a display 112, efficiency level of the user 102, the count of calories burned by the user 102, and the set of user profile attributes. Alternatively, a user device (not shown in FIG. 1) may be used to render the aforementioned data to the user 102. The user device, for example, may be a laptop, a tablet, a notebook, a netbook, a smartphone, a mobile phone, or any other computing device.

It may be apparent that since the total calories burned by the user 102 is determined specifically for a first activity (for example, weighted squats) based on AI based comparison with the target user performing weighted squats, the computation of the calories burned by the user 102 is very accurate and specific for weighted squats. When the same user 102 performs a different second activity (for example, burpees), the AI based comparison is done with the target user performing burpees. Thus, for the same user 102, even when parameters like heart rate or change in heart rate may be same while performing weighted squats or burpees, the total calories burned for each of these activities may be completely different.

Moreover, the current invention works in a synchronous manner along with performance of the activity by the user 102. The AI model 120 may process each of the plurality of frames of the video in real-time or near real-time to instantly determine user efficiency level and calories burned by the user 102 contemporaneous to the user 102 performing the activity. In other words, as the user 102 is performing an activity, the user 102 is constantly and synchronously kept informed with regards to the burned calories and the overall efficiency till that time instant. Based on this instant feedback, the user 102 may also be able to make corrections by improving, for example, the pace, intensity, or range of motion of a particular activity. By way of an example, the AI model 120 determines and compares the current set of activity parameters with target activity parameters contemporaneous to the user 102 performing the activity. Thus, as the AI model 120 detects that a first repetition from a set of an activity is complete, the AI model 120 instantly determines and renders the user efficiency level and the instant calories burned by the user 102. The AI model 120 also displays a cumulative efficiency level and the total calories burned once the set is complete. The constant and accurate feedback provided to the user 102 helps in improving efficiency level of the user 102. Moreover, since the feedback on efficiency is provided specifically with regards to certain activity parameters (for example, range of motion etc.), the user 102 is able to constantly improve his/her performance.

It should be noted that all such aforementioned modules 102-124 may be represented as a single module or a combination of different modules. Further, as will be appreciated by those skilled in the art, each of the modules 102-124 may reside, in whole or in parts, on one device or multiple devices in communication with each other. In some embodiments, each of the modules 102-124 may be implemented as dedicated hardware circuit comprising custom application-specific integrated circuit (ASIC) or gate arrays, off-the-shelf semiconductors such as logic chips, transistors, or other discrete components. Each of the modules 102-124 may also be implemented in a programmable hardware device such as a field programmable gate array (FPGA), programmable array logic, programmable logic device, and so forth. Alternatively, each of the modules 102-124 may be implemented in software for execution by various types of processors (e.g., processor 106). An identified module of executable code may, for instance, include one or more physical or logical blocks of computer instructions, which may, for instance, be organized as an object, procedure, function, or other construct. Nevertheless, the executables of an identified module or component need not be physically located together but may include disparate instructions stored in different locations which, when joined logically together, include the module and achieve the stated purpose of the module. Indeed, a module of executable code could be a single instruction, or many instructions, and may even be distributed over several different code segments, among different applications, and across several memory devices.

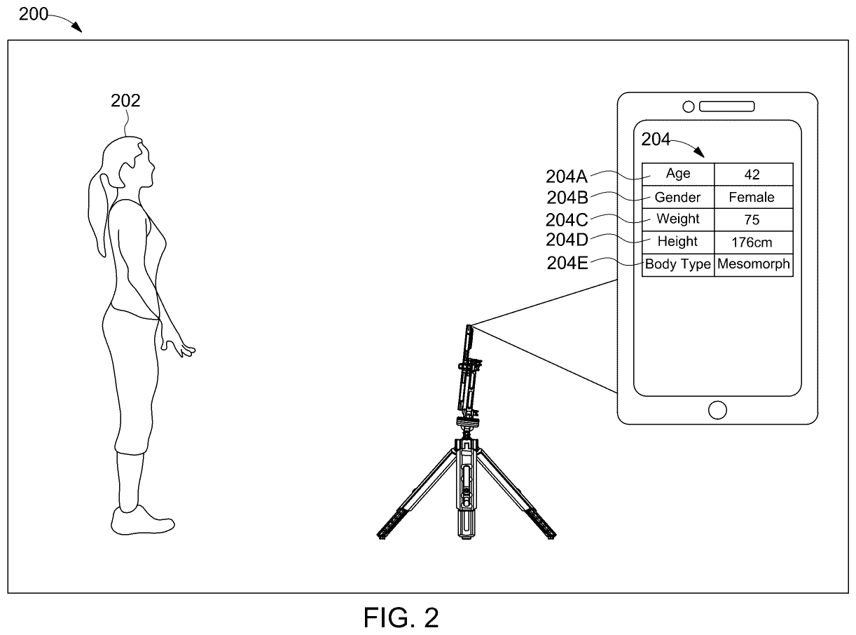

Referring now to FIG. 2, an exemplary scenario where profile attributes of a user 202 are being captured and subsequently displayed via a GUI 200 is illustrated, in accordance with some embodiments. In the current scenario, the user 202 may plan to perform body weight squats. The user 202 may initially stand in front of a computing device (such as the computing device 104) facing an in-built camera (such as the camera 110). The camera may scan the full body image of the user 202. In a scenario, because of an inaccurate posture of the user 202 relative to the computing device, the camera may not be able to scan the full body image of the user 202. In such a case, the GUI 200 may render a message (for example, text message, graphical message, audio message, or visual message) to the user 202. The message may be “Adjust Your Body Posture,” “Posture Reminder,” “Check Body Posture,” “move closer,” “move farther,” or the like. In some embodiments, upon receiving the correct body posture, the GUI 200 may display a message to appreciate the user 202, such as, “Correct Posture—Well Done,” “Nice Work,” “Excellent,” or the like.

Once the image is scanned, the AI model using computer vision (such as the AI model 120) may determine the set of user profile attributes 204 of the user 202. Once the set of profile attributes 204 of the user 202 is detected, the GUI 200 may display one or more of the set of profile attributes. The displayed profile attributes may include age 204A as ‘42’, gender 204B as ‘female’, weight 204C as “75 Kilograms (Kg)’, height 204D as ‘176 centimeters (cm)’, and a body type 204E as ‘mesomorph.’ As discussed before, the set of profile attributes may alternatively be extracted or retrieved from a third-party database that stores such information.

Once the set of user profile attributes are displayed, the GUI 200 may also prompt the user 202 to confirm whether the displayed set of profile attributes 204 of the user 202 is correct or not. The user 202 may accordingly provide a command to confirm or modify the displayed set of profile attributes 204. The command may be provided by the user 202 using at least one of a gesture, a touch, an audio command, or a signal generated by an input device (such as, keyboard, mouse, stylus, graphic pen, or the like). It should be noted that the set of user profile attributes 204 of the user 202 may be stored in a database (such as the database 122) for future reference.

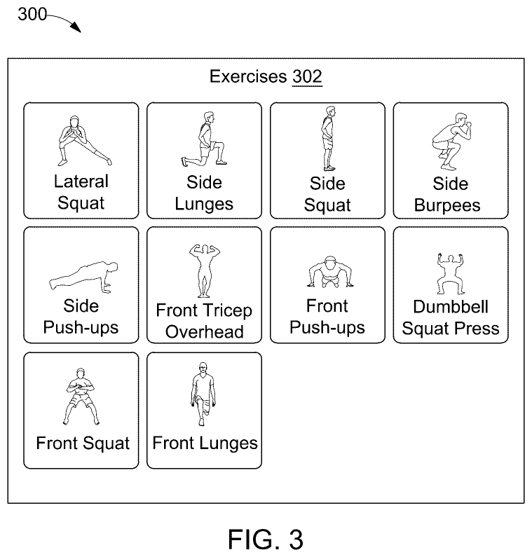

Referring now to FIG. 3, an exemplary GUI 300 displaying user selection of an activity from a plurality of activities 302 is illustrated, in accordance with some embodiments. Each of the plurality of activities 302 may belong to at least one activity category, such as, an aerobic activity, a cardiovascular activity, a strength training activity, boxing, yoga, and the like. The GUI 300 may display the plurality of activities 302 such as lateral squat, side lunges, side squat, side burpees, side pushups, front triceps overhead, push-ups front, dumbbell squat press, squat front, lunges front, and the like.

Once the set of user profile attributes 204 are confirmed, the user 202 may be prompted to start performing any activity. The AI model 120 may automatically detect the activity being performed by the user 102. Alternatively, the AI model 120 may direct the user 202 to perform an activity and once the user 202 starts performing the activity, the AI model 120 may confirm that the user 202 is performing the same activity as directed by the AI model 120 or not. To this end, as the user 202 starts performing an activity, the camera may capture a real-time video stream of the user 202. The real-time video stream may include a plurality of frames, each including a full body image of the user 202 performing the activity. For each of the plurality of frames, the AI model 120 may create a multimedia vector corresponding to the associated frame. Further, the AI model 120 may compare the multimedia vectors created for the plurality of frames with multimedia vectors created for the plurality of activities 302 that are pre-identified. Accordingly, the AI model 120 may identify the activity being performed by the user 202 based on the closest matching activity from the plurality of activities 302. In other words, the AI model may predict or affirm the activity performed by the user 202 in response to processing the real-time video stream.

The identification of the activity from the plurality of activities 302 may be displayed to the user 202 via the GUI 300. In an embodiment, the predicted activity may be highlighted amongst the predefined plurality of exercises 302 on the GUI 300. In some embodiments, the predicted activity may be highlighted with different colours or boundaries on the GUI 300. In some embodiments, once the predicted activity of the user 202 is displayed, the GUI 300 may prompt the user 202 to confirm whether the displayed activity is correct or not. For example, the GUI 300 may render a message to the user 202 such as “Please Verify the Activity,” “Please Check,” “Please Confirm Your Activity,” and the like.

The user 202 may accordingly provide a command to confirm or modify the identified activity. The command may be provided by the user 202 using at least one of a gesture, a touch, an audio command, or a signal generated by an input device (such as, keyboard, mouse, stylus, graphic pen, or the like). If the predicted activity is correct according to the user feedback, the GUI 300 may render a message to the user 202, such as, “The Posture Is Correct,” “Go Ahead,” “Correct Posture,” and the like.

In some embodiments, the activity which will be performed by the user may be received as an input from the user via a user interface (such as the user interface 124). In such a case, the GUI 300 may prompt the user 202 to select one activity from the predefined plurality of activities or may create a circuit that includes multiple activities with different repetitions and durations. In such a case, the user 202 may be required to manually select an activity from the plurality of activities 302 before initiating performance of the activity. In some embodiments, the plurality of activities 302 may be sorted based on one of the sorting criteria. By way of an example, the sorting criteria may include, but may not be limited to new exercises, latest exercises performed, most frequent exercises performed, and duration of an exercise. It may be noted that the GUI 300 may not be limited to fitness and may be customized based on user requirements and use cases. For example, the GUI 300 may include activities for specific therapies in case of rehab, meditations for Yoga, physiotherapy recommended by a medical professional, or the like. In continuation of the above example, the user 202 may select one activity from the plurality of activities 302, for example, front squat. The user may select the activity (i.e., front squat) using at least one of a gesture, a touch, an audio command, or a signal generated by an input device (such as, keyboard, mouse, stylus, graphic pen, or the like).

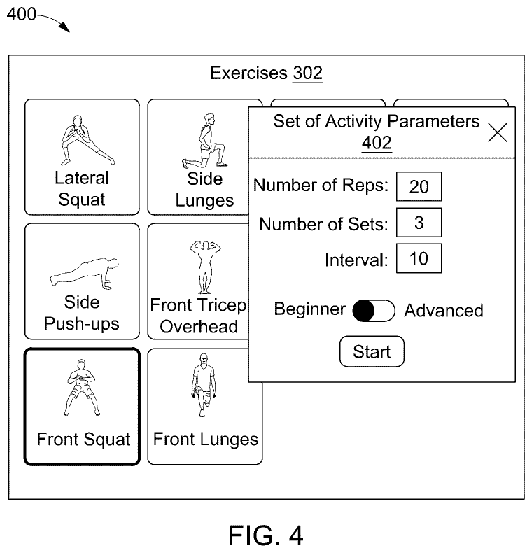

Referring now to FIG. 4, an exemplary GUI 400 displaying selection of an activity and corresponding activity parameters 402 is illustrated, in accordance with some embodiments. Upon receiving selection of the activity from the user 202, the GUI 400 may request the user 202 to provide the set of activity parameters 402. By way of an example, the set of activity parameters 402 may include, but may not be limited to number of reps (e.g., 20), number of sets (e.g., 3), interval (e.g., every 10 seconds), and a level of exercise (for example, beginner or advanced).

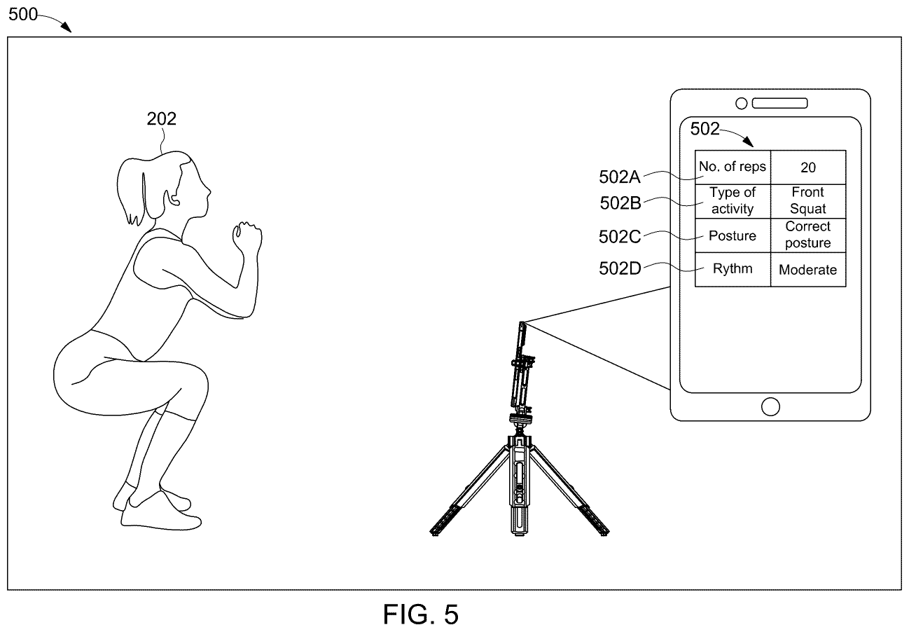

Referring now to FIG. 5, an exemplary scenario of the user 202 performing the selected activity from the computing device 104 and a GUI 500 displaying a current set of activity parameters of the user 202 is illustrated, in accordance with some embodiments. As discussed above, the user 202 has selected front squats as the activity that the user 202 intends to perform in front of the computing device. The camera may capture the real-time video stream of the user 202 performing the front squats. The real-time video stream of the user 202 may include a plurality of frames. The AI model 120 may process the plurality of frames and may create a multimedia vector for each of the plurality of frames. The AI model 120 may then process the multimedia vectors to determine the current set of activity parameters of the user 202. This has already been explained in detail in the FIG. 1 given above. By way of an example, the GUI 500 may display the following set of activity parameters 502 of the user 202: the number of reps 502A as “20,” a type of activity 502B as “front squats,” a posture 504C as “correct posture,” and a rhythm 504D as “moderate.”

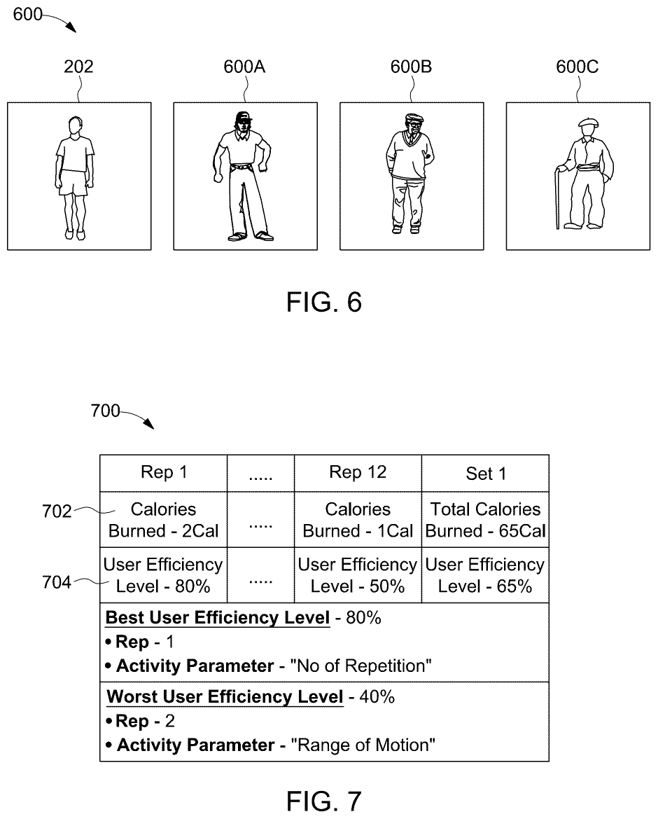

Referring now to FIG. 6, an exemplary scenario of selection of a target user matching with the current user 202 from amongst a plurality of target users is illustrated, in accordance with some embodiments. In the current embodiment, profile attributes of the user 202 may be age—42 years, gender—male, weight—75 Kg, height—176 cm, and body type—mesomorph. As depicted in FIG. 6, profile attributes of the user 202 are compared with three target users, i.e., target users 600A, 600B, and 6000. Profile attributes of the target user 600A may be age—42 years, gender—male, weight—75 Kg, height—176 cm, and a body type—mesomorph. In a similar manner, profile attributes of the target user 600B may be age—40 years, gender—male, weight—85 Kg, height—171 cm, and a body type—endomorph. Further, profile attributes of the target user 6000 may be age—60 years, gender—male, weight—70 Kg, height—170 cm, and a body type—endomorph.

The AI model 120 may select one of the target users 600A, 600B, or 6000 based on similarity of their profile attributes with profile attributes 204 of the user 202. For a given set of profile attributes and the front squat exercise, the selected target user is a benchmark user. In order to select the target user, a similarity score between the profile attributes of the user 202 and the target users may be calculated for each of the target users 600A, 600B, and 6000. Because of the degree of similarity between profile attributes of the user 202 and the target user 600A is high, the similarity score calculated for the target user 600A is the highest. Thus, the target user 600A is selected from amongst the target user 600A, 600B, and 6000. Further, upon selection of the target user 600A, target activity parameters associated with the target user 600A may be identified. As discussed before, the target activity parameters of the target user 600A for front squats as an activity may already be stored in the database 122. The activity parameters of the target user 600A for front squats may be, pace—“30 squats per minute,” the number of repetitions—“3 sets of 25 squats,” and the like. For these set of activity parameters, the target user 600A may burn 100 calories while performing front squats. These target activity parameters may then be used to determine efficiency level and the total calories burned by the user 202. This is further explained in detail in conjunction with FIG. 7.

Referring now to FIG. 7, an exemplary GUI 700 displaying performance details of the user 202 performing the selected activity is illustrated, in accordance with some embodiments. When the user 202 performs front squats, the computing device 104 may first determine activity parameters of the user 202. This is already depicted and described in FIG. 5.

Thereafter, the activity parameters of the user 202 may be compared with target activity parameters of the target user 600A as discussed in FIG. 6. In continuation with the example given in FIG. 5, the pace of squats as performed by the user 202 is “15 squats per minute,” while the pace (i.e., target pace) of squats performed by the target user 600A is “30 squats per minute.” The efficiency level of the user 202 for this specific activity parameter is 50%. Similarly, the number of repetitions of squats performed by the user 202 is “3 sets of 20 squats,” while the number of repetitions of squats performed by the target user 600A is “3 sets of 25 squats.” The efficiency level of the user 202 for this specific activity parameter is 50%. In another similar example, the range of motion of squats performed by the user 202 is may be partial, while the range of motion of the target user 600A may be full. The efficiency level of the user 202 for this specific activity parameter may be 50% or less. Based on the comparison, the cumulative efficiency level of the user 202 may be determined as ‘65%’. This cumulative efficiency level may be a simple average; however, it may be noted that a more complex function may be used to arrive at the overall efficiency level of the user 202.

Since the total number of calories burned by the target user was 100 calories, the total calories burned by the user 202 would be 65% of 100 calories, i.e., 65 calories. It will be apparent that the examples given above are used for ease of explanation, however, a linear mapping as explained in the above example either for computation of the efficiency level or for the burned calories may not be actually implemented. Adjustments in the efficiency of the user 202 and the calories burned may be performed statistically, Algorithmically, or by using AI. By way of an example, 65% less efficiency of the user 202 may actually translate to a 10% difference in the calorie burn rate or the total calories burned by the user 202.

In some embodiments, the AI model 120 may fail to find a target user who has same or similar profile attributes as that of the user 202. In such a case, a benchmark user whose profile attributes and activity parameters for squats are available in the database 122 may be selected as the target user. The user efficiency level may then be determined by comparing user activity parameters with target activity parameters associated with the benchmark user. By way of an example, the user efficiency level may be determined as 50%. Profile attributes of the user 202 may then be compared with benchmark profile attributes of this benchmark user. Further, a deviation of one or more profile attributes of the user 202 when compared to profile attributes of the benchmark user may be computed. By way of an example, the deviation of the profile attributes of the user 202 may be 30%. In other words, in terms of profile attributes, the user 202 may be 30% less capable when compared to the benchmark user for performing squats. Based on the computed deviation, the user efficiency level may now be adapted (or modified). In continuation of the example given above, the user efficiency level may be increased by 30% to arrive at an adjusted user efficiency level of 65% (i.e., 1.3*50%). If the calories burned by the benchmark user for performing squats was 100 Calories, the total calories burned by the user 202 would be 65% of 100 calories, i.e., 65 Calories. It will be apparent that the examples given above are used for ease of explanation, however, a linear mapping as explained in the above example either for computation of the efficiency level or for the burned calories may not be actually implemented. Adjustments in the efficiency of the user 202 and the calories burned may be performed statistically, Algorithmically, or by using AI.

Irrespective of the method used to arrive at the final user efficiency level of the user 202, the GUI 700 may display calories burned 702 by the user 202 as 65 Calories (total calories for Set 1) and the user efficiency level 704 as 65% (cumulative user efficiency level for Set 1). In some embodiments, the GUI 700 may display one or more message that may include “Congratulations”, “Goal Achieved”, or “Keep It Up,” when either of the user efficiency level or total calories burned may exceed a predefined limit.

It may further be noted that the GUI 700, in real-time, may display the user efficiency level and the calories burned 702 by the user 202 contemporaneous to the user 202 performing the activity. In other words, the GUI 700 may instantly display the current user efficiency level and the current calories burned by the user 202 per repetition within a set of the activity. By way of an example, the user 202 may have selected 3 sets of front squats with 12 repetitions in each set. Once the user 202 completes the first repetition of front squat within the first set, the GUI 700 may immediately display the calories burned 702 and the user efficiency level 704 for the first repetition. As depicted in FIG. 7, for “Rep 1” the GUI 700 may display the calories burned 702 by the user 202 as 2 Calories, and the user efficiency level 704 as 80%. Similarly, for “Rep 12” the GUI 700 may display the calories burned 702 by the user as 1 Calorie and the user efficiency level 704 as 50%. In a similar manner, the GUI 700 may display the calories burned 702 and the user efficiency level 704 by the user 202 for remaining repetitions. Additionally, the GUI 700 may also display the total calories burned 702 by the user as 65 Calories and the total user efficiency level 704 as 65%. It should be noted that the total calories burned 702 and the total user efficiency level 704 may be an average of all 12 repetitions performed by the user 202 in “Set 1.” Thus, the calories burned 702 and the user efficiency level 704 may be updated for the user 202 on the GUI 700 in real-time, for each subsequent repetition and each subsequent set.

In some embodiments, the GUI 700 may also indicate the best repetition and/or set performed by the user in terms of calories burned and/or efficiency level. Additionally, the GUI 700 may also indicate a specific activity parameter where the efficiency level of the user 202 was the best or worst. As depicted and in continuation with the above example, the GUI 700 may indicate that the ‘Best User Efficiency Level” was 80%. The GUI 700 may also further indicate that the relevant repetition for this efficiency level was “Rep 1,” and the relevant activity parameter was “Number of Repetitions.” Additionally, the GUI 700 may indicate that the ‘Worst User Efficiency Level” was 40%. The GUI 700 may also further indicate that the relevant repetition for this efficiency level was “Rep 2,” and the relevant activity parameter was “Range of motion.”

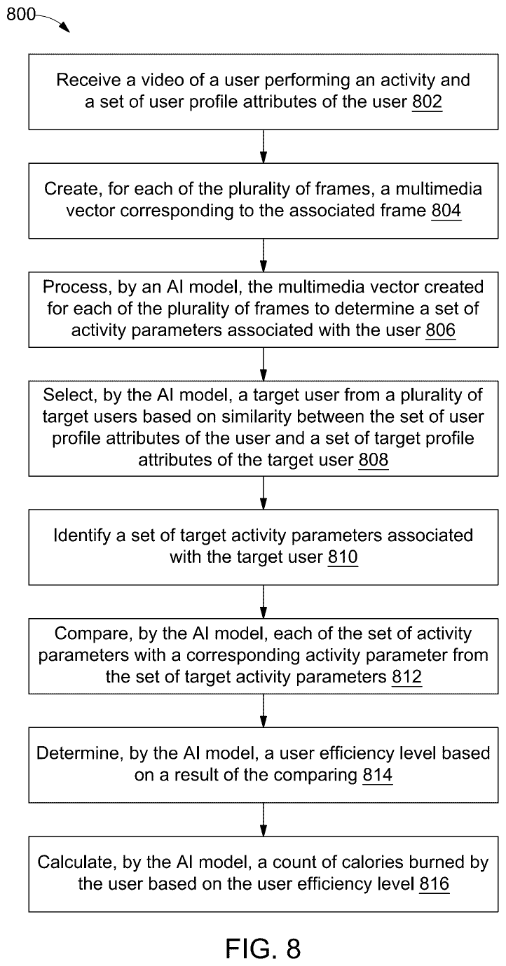

Referring now to FIG. 8, an exemplary process 800 for calculating calories burned during activity performance using AI is illustrated via a flowchart, in accordance with some embodiments. FIG. 8 is explained in conjunction with FIG. 1 to FIG. 7. The process 800 may be implemented by the computing device 104. In some embodiments, the process 800 may include receiving a video stream (such as, a real-time video, near real-time video, or a pre-recorded video) of a user (such as the user 102) performing an activity and a set of user profile attributes of the user, at step 802. It should be noted that the video may include a plurality of frames. The set of user profile attributes, for example, may include, but are not limited to age, gender, weight, height, body type, and the like. The video stream of the user while performing the activity may be captured through a camera (such as the camera 110). In some embodiment, the set of user profile attributes may automatically be determined by a computing device (such as the computing device 104). To this end, initially, the computing device may instruct the user, via a user interface (such as the user interface 124) to stand in front of the camera facing forward and then facing backwards. Further, the camera may start capturing (or scanning) a full body image of the user to determine the set of user profile attributes. Further, upon scanning the image of the user, an AI model (such as the AI model 120) may process the scanned image of the user using a computer vision technology to determine the set of user profile attributes. In some embodiments, the set of user profile attributes may be received from the user as an input via the user interface. In some embodiments, the video of the user while performing the activity may be captured through a camera that is external to the computing device 104. Further, the video may be uploaded to the computing device via the user.

Once the video stream and the set of user profile attributes of the user is received, the process 800 may include creating, for each of the plurality of frames, a multimedia vector corresponding to the associated frame, at step 804.

Upon creating the multimedia vector, the process 800 may include processing, by an AI module (such as the AI module 118), the multimedia vector created for each of the plurality of frames via the AI model to determine a set of activity parameters associated with the user, at step 806. By way of an example, the set of activity parameters may include, but may not be limited to, a type of activity, a pace of performing the activity, number of repetitions of the activity, an accuracy of performing the activity, a degree of freedom of the activity being performed, periodicity (i.e., rhythm) of the activity, range of motion, duration for which the activity was performed, and the like.

In some embodiments, the activity which is being performed by the user may be identified by the AI model using a computer vision technology. The AI model may automatically detect the activity being performed by the user. Alternatively, the AI model may direct the user to perform an activity and once the user starts performing the activity, the AI model may confirm that the user 202 is performing the same activity as directed by the AI model 120. To this end, initially, the AI model may receive in real-time the video stream of the user. The video stream may include the plurality of frames, each including the full body image of the user performing the activity.

Once the video stream is received, the AI model may create, for each of the plurality of frames, a multimedia vector corresponding to the associated frame. The AI model may then compare the multimedia vector created for the plurality of frames to a multimedia vector created for a plurality of activities that are pre-defined. It should be noted that the plurality of activities may be pre-stored in a database (such as the database 122). Accordingly, the AI model may identify the activity being performed by the user based on the closest matching activity from the plurality of activities. In other words, the AI model may predict or affirm the activity performed by the user in response to processing the real-time video stream.

When the user starts performing any activity and the AI model predicts or identifies the activity being performed, the AI model may render a description associated with the prediction to the user. The user may then confirm whether the activity prediction is correct or not. In case the activity prediction is incorrect as per the user confirmation, the AI model may provide alternate prediction or may prompt the user to either select the relevant activity from a plurality of options or directly provide details of the activity being performed.

By way of an example, consider a scenario where the user may decide or may be instructed by the AI model to perform ‘push-ups.’ Before the user initiates performance of push-ups, the set of user profile attributes of the user may automatically be determined by the computing device. To determine the set of user profile attributes, the computing device may instruct the user to stand in front of camera (e.g., facing forward or facing backward). Further, the camera may start capturing the full body image of the user while performing the push-ups. Further, once the image is captured (or scanned), the AI model may process the captured image to determine the set of user profile attributes using the computer vision technology. For example, in current scenario, the set of user profile attributes of the user may be, for example, age—45 years, weight—85 kilograms (Kg), gender—male, height—171 centimetres, and body type—mesomorph. Some historic profile attributes of the user, for example, physical abilities of the user, such as, balance, coordination, or speed, may also be retrieved from a database.

Once the set of user profile attributes have been determined, the user may then start performing the push-ups. While the user is performing the push-ups, the video stream of the user may be captured in real-time through the camera. The video stream may include the plurality of frames. For each of the plurality of frames, multimedia vectors may be created which may then be processed by the AI model to determine the set of activity parameters associated with the user for push-ups. In the current example, the activity parameters may be determined as a type of the activity—body weight exercise, the pace as (15 push-ups per minute, number of repetitions as 30 push-ups, accuracy with respect to posture as 80%, range of motion as partial, rhythm as moderate, and total duration of performing push-ups as 2 minutes.

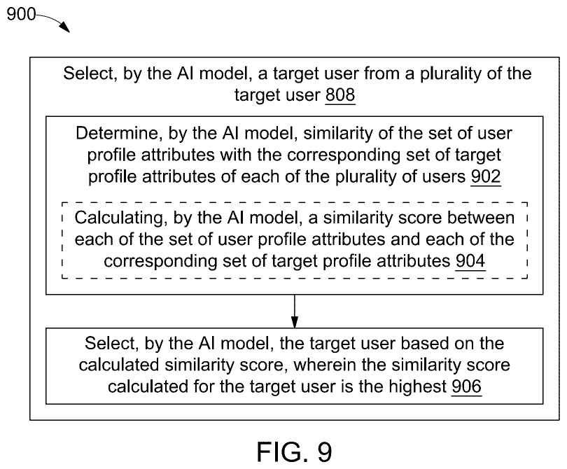

Once the set of activity parameters is determined, the process 800 may include selecting, by the AI model, a target user from a plurality of target users based on similarity between the set of user profile attributes of the user and a set of target profile attributes of the target user, at step 808. The target user may correspond to a benchmark user. A benchmark user is a user whose profile attributes and activity parameters for the specific activity that user is currently performing may be available in the database. As will be appreciated, the target user may be any person who has been clinically and/or accurately monitored for calorie expenditure estimation for at least one activity. By way of an example, the target user with the closest similarity score with the user may be selected as the target user. This is further explained in greater detail in conjunction with FIG. 9. In continuation with the above example, the set of profile attributes of the target user may be age—45 years, weight—85 kilograms (Kg), gender—male, height—171 centimetres, and body type—mesomorph. It should be noted that the set of profile attributes of the target user may already be stored in the database.

Once the target user is selected, the process 800 may include identifying, via the AI model, a set of target activity parameters associated with the target user, at step 810. By way of an example, the set of target activity parameters of the target user may include, but may not be limited to a type of activity, a pace of performing the activity, number of repetitions of the activity, an accuracy of performing the activity, a degree of freedom of the activity being performed, periodicity (i.e., rhythm) of the activity, range of motion, duration for which the activity was performed, and the like. The set of target activity parameters associated with the target user may already be stored in the database. Once the target user is selected, the process 800 may include retrieving the set of target activity parameters associated with the target user from the database. This is further explained in greater detail in conjunction with FIG. 9. In continuation with the above example, the set of target activity parameters for push-ups may be the pace as 30 push-ups in a minute), number of repetitions as 60 reps), an accuracy with respect to posture as 90%, rhythm as fast, and total duration as 2 minutes.

The process 800 may further include comparing, by the AI model, each of the set of activity parameters with a corresponding activity parameter from the set of target activity parameters in real-time contemporaneous to the user performing the activity, at step 812. By way of an example, the set of activity parameters associated with the push-ups being performed by the user may be compared with the set of target activity parameters associated with the target activity for push-ups. For example, the accuracy of the push-ups being performed by the user may be compared with the accuracy of the push-ups performed by the target user. Similarly, the total number of push-ups done by the user may be compared with the total number of push-ups performed by the target user. As will be appreciated, in a similar manner, each of the set of activity parameters may be compared with a corresponding activity parameter associated with the target user.

Further, the process 800 may include determining a user efficiency level based on a result of the comparing, at step 814. The user efficiency level may be determined by the AI model. Alternatively, various algorithmic or statistical models may be used to determine the user efficiency level. In continuation of the above example, the pace of push-ups as performed by the user is “15 push-ups per minute”, while the target pace is “30 push-ups per minute”. Upon comparison, the user efficiency level may be determined as 50%.

It will be apparent that the current example is used for ease of explanation, however, a linear mapping as explained in the above example may not be actually implemented. Adjustments in the efficiency of the user may be performed statistically, Algorithmically, or by using AI. By way of an example, 75% less efficiency of the user may actually translate to a 20% difference in the calorie burn rate or the total calories burned by the user.

Once the user efficiency level is determined, the process 800 may include calculating, by the AI model, a count of calories burned by the user based on the user efficiency level, at step 816. In continuation of the above example, the user efficiency level is determined as 50%. Since the count of calories burned by the target user while performing the push-ups for the same time duration was 400 calories, the count of calories burned by the user would be 200 calories (i.e., 50%*400 calories).

Further, once the count of calories burned by the user is calculated, the process 800 may include rendering via a GUI, the efficiency level of the user (i.e., 55%), the count of calories burned by the user (i.e., 200 Calories), and the set of user profile parameters on a user device (e.g., a smartphone).

The current invention works in a synchronous manner along with performance of the activity by the user. The AI model may process each of the plurality of frames video in real-time or near real-time to instantly determine user efficiency level and calories burned by the user contemporaneous to the user performing the activity. In other words, as the user is performing an activity, the user is constantly and synchronously kept informed with regards to the burned calories and the overall efficiency till that time instant. Based on this instant feedback, the user may also be able to make corrections by improving, for example, the pace, intensity, or range of motion of a particular activity. By way of an example, as the AI model determines and compares the current set of activity parameters with target activity parameters contemporaneous to the user performing the activity, as the AI model detects a first repetition from a set of an activity is complete, the AI model instant determines and renders the user efficiency level and the instant calories burned by the user. The AI model also displays a cumulative efficiency level and the total calories burned once the set is complete. The constant and accurate feedback provided to the user helps in improving efficiency level of the user, moreover, since the feedback on efficiency is provided specifically with regards to certain activity parameters (for example, range of motion etc.), the user is able to constantly improve his/her performance.

Referring now to FIG. 9, an exemplary process for selecting a target user from a plurality of target users is illustrated via a flowchart, in accordance with some embodiments. FIG. 9 is explained in conjunction with FIG. 8. The process 900 may include selecting, by an AI model (such as the AI model 120) the target user from the plurality of target users based on similarity between a set of user profile attributes of a user and a set of target profile attributes of the target user, at step 808. The step 808 of the process 900 may include determining, by the AI model, similarity of the set of user profile attributes with the corresponding set of target profile attributes of each of the plurality of target users, at step 902. Further, the step 902 of the process 900 may include calculating, by the AI model, a similarity score between each of the set of user profile attributes and each of the corresponding set of target profile attributes, at step 904. It should be noted that the similarity score may be calculated by the AI model. Alternatively, the similarity score may be determined using one of Euclidean distance, Manhattan distance, Minkowski distance, and Chebyshev distance. Further, upon calculating the similarity score, the process 900 may include selecting, by the AI model, the target user based on the calculated similarity score, at step 906. The similarity score calculated for the target user is the highest.

By way of an example, consider the scenario where the details of the two target users may be available in the database. A target user ‘1’ may have a set of target profile attributes such as age—45 years, weight—85 kg, height—171 cm, gender—male, body type—mesomorph, and the like that may already be stored in the database. Similarly, a target user ‘2’ may have the set of target profile attributes as age—65 years, weight—75 kg, height—165 cm, gender—male, body type—ectomorph, and the like that may also be already stored in the database.

Once the set of activity parameters of the user is determined, the AI model may select the target user from the plurality of target users. To this end, the AI model may determine the similarity of the set of user profile attributes with the corresponding set of target profile attributes of each of the plurality of target users. Further, the AI model may calculate the similarity score between each of the set of user profile attributes and each of the corresponding set of target profile attributes (i.e. for the target user ‘1’) using a similarity analysis (e.g., cosine distance). By way of an example, the similarity score for the target user ‘1’ may be ‘0.8’, and the similarity score for the target user ‘2’ may be ‘0.6’. It should be noted that the similarity score range may be between ‘0-1’. Upon calculating the similarity score for each of the two target users, one target user may be selected based on the calculated similarity score. The target user may be selected whose similarity score is highest among the two target users. Thus, the target user ‘1’ is selected as the target user in the current example.

Referring now to FIG. 10, an exemplary process for identifying a set of target activity parameters is illustrated via a flow chart, in accordance with some embodiments. FIG. 10 is explained in conjunction with FIG. 8 and FIG. 9. In some embodiments, the process 1000 may include identifying, by an AI model (such as the AI model 120), the set of target activity parameters associated with the target user, at step 810. The step 810 of the process 1000 may include receiving a video (or video stream) of the target user performing a target activity (for example, push-up) and a set of target profile attributes of the target user, at step 1002. The set of target profile attributes may include, but may not be limited to, age, gender, weight, height, body type, and other physical abilities. The video stream may include a plurality of frames. The video stream of the target user may be captured using a camera.

Once the video stream and the set of profile attributes associated with the target user is received, the process 1000 may include creating, for each of the plurality of frames, a multimedia vector corresponding to the associated frame, at step 1002. Upon creating the multimedia vector, the process 1000 may include processing, by an AI model (such as the AI model 120), the multimedia vector created for each of the plurality of frames to determine the set of target activity parameters associated with the target user, at step 1004.

Further, once the set of target activity parameters is determined, the process 1000 may include determining an estimated target calories for the target activity based on a count of calories burned by the target user corresponding to the target activity, at step 1006. Further, once the estimated target calories are determined, the process 1000 may include setting the target efficiency level corresponding to the target activity, at step 1008. It should be noted that the target efficiency level of the target user may be 100% for the target activity. Once the target efficiency level is computed, the process 1000 may include storing, by the AI model, the set of target activity parameters associated with the target user in a database (such as the database 122), at step 1010. Additionally, the process 1000 may include storing, by the AI model, the target efficiency level within the database, at step 1012. Thus, once the target user is selected, the set of target activity parameters, calories burned, and target efficiency level associated with the target user from the database.

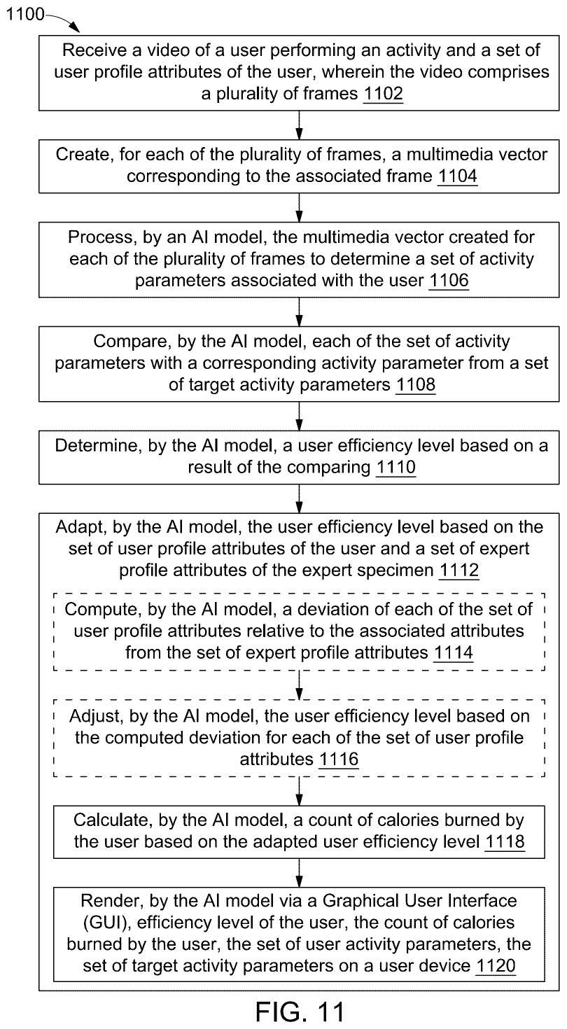

Referring now to FIG. 11, another exemplary process for calculating calories burned during activity performance using AI is illustrated via a flow chart, in accordance with some embodiments. FIG. 11 is explained in conjunction with FIG. 1 and FIG. 8-FIG. 10. The process 1100 may be implemented by the computing device 104 of the system 100. In some embodiments, the process 1100 may include receiving a video stream of a user performing an activity and a set of user profile attributes of the user, at step 1102. The video stream may include a plurality of frames. The video stream of the user while performing the activity may be captured using a camera (such as the camera 110).

Once the video stream and the set of profile attributes of the user is received, the process 1100 may include creating, for each of the plurality of frames, a multimedia vector corresponding to the associated frame, at step 1104. Upon creating the multimedia vector, the process 1100 may include processing, by the AI model, the multimedia vector created for each of the plurality of frames to determine a set of activity parameters associated with the user, at step 1106. The set of activity parameters may be, for example, but may not be limited to, a type of activity, a pace of performing the activity, number of repetitions of the activity, an accuracy of performing the activity, a degree of freedom of the activity being performed, range of motion, periodicity (i.e., rhythm) of the activity, duration for which the activity was performed, and the like.

Once the set of activity parameters is determined, the process 1100 may include comparing, by the AI model, each of the set of activity parameters with a corresponding activity parameter from a set of target activity parameters associated with a benchmark user in real-time contemporaneous to the user, at step 1108. The set of target activity parameters may correspond to the activity being performed by the benchmark user.

Upon comparison, the process 1100 may include determining, by the AI model, a user efficiency level based on a result of the comparing, at step 1110. Once the user efficiency level is determined, the process 1100 may include adapting, by the AI model, the user efficiency level based on the set of user profile attributes of the user and a set of benchmark profile attributes of the benchmark user, at step 1112. Further, in order to adapt the user efficiency level, the process 1100 may include computing, by the AI model, a deviation of each of the set of user profile attributes relative to the associated attributes from the set of benchmark profile attributes, at step 1114. Further, the process 1100 may include adjusting, by the AI model, the user efficiency level based on the computed deviation for each of the set of user profile attributes, at step 1116.

By way of an example, the user efficiency level may be adapted based on the set of user profile attributes and the set of benchmark profile attributes. A deviation of each of profile attributes of the user when compared to profile attributes of the benchmark user may be computed to be 30%. Thus, in terms of profile attributes, the user 202 may be 30% less capable when compared to the benchmark user for performing burpees. Therefore, the adapted user efficiency level may be increased by 30% to arrive at an adjusted user efficiency level of 91% (i.e., 1.3*70%). It will be apparent that the current example is used for ease of explanation, however, a linear mapping as explained in the above example may not be actually implemented. Adjustments in the efficiency of the user may be performed statistically, Algorithmically, or by using AI. By way of an example, 75% less efficiency of the user may actually translate to a 10% difference in the calorie burn rate or the total calories burned by the user.

Once the user efficiency level is adapted, the process 1100 may include calculating, by the AI model, a count of calories burned by the user based on the adapted user efficiency level, at step 1118. By way of an example, if the count of calories burned by the benchmark user is 100 calories, the count of calories burned by the user may be 91 calories. Upon calculating the count of calories, the process 1100 may include rendering via a GUI, the user efficiency level of the user, the set of user profile attributes of the user, and the count of calories burned by the user on a user device, at step 1120.

The current invention works in a synchronous manner along with performance of the activity by the user. The AI model may process each of the plurality of frames video in real-time or near real-time to instantly determine user efficiency level and calories burned by the user contemporaneous to the user performing the activity. In other words, as the user is performing an activity, the user is constantly and synchronously kept informed with regards to the burned calories and the overall efficiency till that time instant. Based on this instant feedback, the user may also be able to make corrections by improving, for example, the pace, intensity, or range of motion of a particular activity. By way of an example, as the AI model determines and compares the current set of activity parameters with target activity parameters contemporaneous to the user performing the activity, as the AI model detects a first repetition from a set of an activity is complete, the AI model instant determines and renders the user efficiency level and the instant calories burned by the user. The AI model also displays a cumulative efficiency level, and the total calories burned once the set is complete. The constant and accurate feedback provided to the user helps in improving efficiency level of the user, moreover, since the feedback on efficiency is provided specifically with regards to certain activity parameters (for example, range of motion etc.), the user is able to constantly improve his/her performance.

As will be also appreciated, the above-described techniques may take the form of computer or controller implemented processes and apparatuses for practicing those processes. The disclosure can also be embodied in the form of computer program code containing instructions embodied in tangible media, such as floppy diskettes, solid state drives, CD-ROMs, hard drives, or any other computer-readable storage medium, wherein, when the computer program code is loaded into and executed by a computer or controller, the computer becomes an apparatus for practicing the invention. The disclosure may also be embodied in the form of computer program code or signal, for example, whether stored in a storage medium, loaded into and/or executed by a computer or controller, or transmitted over some transmission medium, such as over electrical wiring or cabling, through fiber optics, or via electromagnetic radiation, wherein, when the computer program code is loaded into and executed by a computer, the computer becomes an apparatus for practicing the invention. When implemented on a general-purpose microprocessor, the computer program code segments configure the microprocessor to create specific logic circuits.

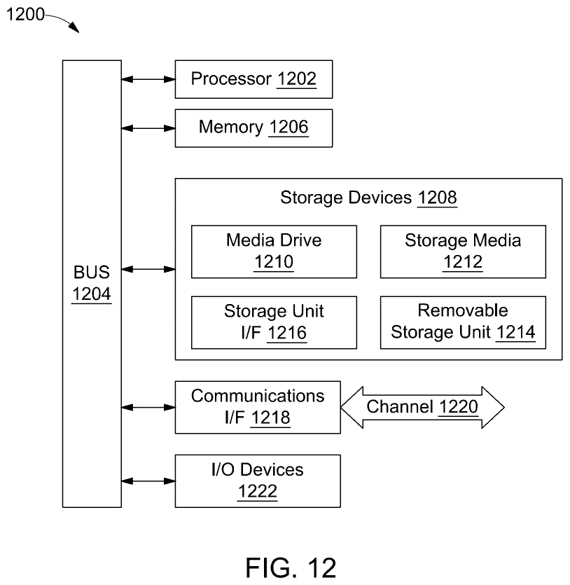

The disclosed methods and systems may be implemented on a conventional or a general-purpose computer system, such as a personal computer (PC) or server computer. Referring now to FIG. 12, an exemplary computing system 1200 that may be employed to implement processing functionality for various embodiments (e.g., as a SIMD device, client device, server device, one or more processors, or the like) is illustrated. Those skilled in the relevant art will also recognize how to implement the invention using other computer systems or architectures. The computing system 1200 may represent, for example, a user device such as a desktop, a laptop, a mobile phone, personal entertainment device, DVR, and so on, or any other type of special or general-purpose computing device as may be desirable or appropriate for a given application or environment. The computing system 1200 may include one or more processors, such as a processor 1202 that may be implemented using a general or special purpose processing engine such as, for example, a microprocessor, microcontroller or other control logic. In this example, the processor 1202 is connected to a bus 1204 or other communication medium. In some embodiments, the processor 1202 may be an Artificial Intelligence (AI) processor, which may be implemented as a Tensor Processing Unit (TPU), or a graphical processor unit, or a custom programmable solution Field-Programmable Gate Array (FPGA).

The computing system 1200 may also include a memory 1206 (main memory), for example, Random Access Memory (RAM) or other dynamic memory, for storing information and instructions to be executed by the processor 1202. The memory 1206 also may be used for storing temporary variables or other intermediate information during execution of instructions to be executed by the processor 1202. The computing system 1200 may likewise include a read only memory (“ROM”) or other static storage device coupled to bus 1204 for storing static information and instructions for the processor 1202.

The computing system 1200 may also include a storage devices 1208, which may include, for example, a media drive 1210 and a removable storage interface. The media drive 1210 may include a drive or other mechanism to support fixed or removable storage media, such as a hard disk drive, a floppy disk drive, a magnetic tape drive, an SD card port, a USB port, a micro USB, an optical disk drive, a CD or DVD drive (R or RW), or other removable or fixed media drive. A storage media 1212 may include, for example, a hard disk, magnetic tape, flash drive, or other fixed or removable medium that is read by and written to by the media drive 1210. As these examples illustrate, the storage media 1212 may include a computer-readable storage medium having stored therein particular computer software or data.

In alternative embodiments, the storage devices 1208 may include other similar instrumentalities for allowing computer programs or other instructions or data to be loaded into the computing system 1200. Such instrumentalities may include, for example, a removable storage unit 1214 and a storage unit interface 1216, such as a program cartridge and cartridge interface, a removable memory (for example, a flash memory or other removable memory module) and memory slot, and other removable storage units and interfaces that allow software and data to be transferred from the removable storage unit 1214 to the computing system 1200.

The computing system 1200 may also include a communications interface 1218. The communications interface 1218 may be used to allow software and data to be transferred between the computing system 1200 and external devices. Examples of the communications interface 1218 may include a network interface (such as an Ethernet or other NIC card), a communications port (such as for example, a USB port, a micro USB port), Near field Communication (NFC), etc. Software and data transferred via the communications interface 1218 are in the form of signals which may be electronic, electromagnetic, optical, or other signals capable of being received by the communications interface 1218. These signals are provided to the communications interface 1218 via a channel 1220. The channel 1220 may carry signals and may be implemented using a wireless medium, wire or cable, fiber optics, or other communications medium. Some examples of the channel 1220 may include a phone line, a cellular phone link, an RF link, a Bluetooth link, a network interface, a local or wide area network, and other communications channels.

The computing system 1200 may further include Input/Output (I/O) devices 1222. Examples may include, but are not limited to a display, keypad, microphone, audio speakers, vibrating motor, LED lights, etc. The I/O devices 1222 may receive input from a user and also display an output of the computation performed by the processor 1202. In this document, the terms “computer program product” and “computer-readable medium” may be used generally to refer to media such as, for example, the memory 1206, the storage devices 1208, the removable storage unit 1214, or signal(s) on the channel 1220. These and other forms of computer-readable media may be involved in providing one or more sequences of one or more instructions to the processor 1202 for execution. Such instructions, generally referred to as “computer program code” (which may be grouped in the form of computer programs or other groupings), when executed, enable the computing system 1208 to perform features or functions of embodiments of the present invention.

In an embodiment where the elements are implemented using software, the software may be stored in a computer-readable medium and loaded into the computing system 1200 using, for example, the removable storage unit 1214, the media drive 1210 or the communications interface 1218. The control logic (in this example, software instructions or computer program code), when executed by the processor 1202, causes the processor 1202 to perform the functions of the invention as described herein.

Various embodiments provide method and system for calculating calories burned during activity performance using AI. The disclosed method and system may receive a video of a user performing an activity and a set of user profile attributes of the user. The video may include a plurality of frames. Further, the disclosed method and system may create, for each of the plurality of frames, a multimedia vector corresponding to the associated frame. Further, the disclosed method and system may process, by an AI model, the multimedia vector created for each of the plurality of frames to determine a set of activity parameters associated with the user. Further, the disclosed method and system may select, by the AI model, a target user from a plurality of target users based on similarity between the set of user profile attributes of the user and a set of target profile attributes of the target user. The target user corresponds to a benchmark user. Further, the disclosed method and system may identify a set of target activity parameters associated with the target user. Further, the disclosed method and system may compare, by the AI model, each of the set of activity parameters with a corresponding activity parameter from the set of target activity parameters. Moreover, the disclosed method and system may determine, by the AI model, a user efficiency level based on a result of the comparing. Thereafter, the disclosed method and system may calculate, by the AI model, a count of calories burned by the user based on the user efficiency level.

Thus, the current invention works in a synchronous manner along with performance of the activity by a user. The AI model may process each of the plurality of frames video in real-time or near real-time to first identify the activity being performed by the user accurately. The AI model then instantly determines user efficiency level and calories burned by the user contemporaneous to the user performing the activity. In other words, as the user is performing an activity, the user is constantly and synchronously kept informed with regards to the burned calories and the overall efficiency till that time instant. Based on this instant feedback, the user may also be able to make corrections by improving, for example, the pace, intensity, or range of motion of a particular activity. The constant and accurate feedback provided to the user helps in improving efficiency level of the user. Moreover, since the feedback on efficiency is provided specifically with regards to certain activity parameters, the user is able to constantly improve his/her performance. This is a vast improvement over existing applications and wearables that neither specify an individual's accuracy in performing a given activity nor provide real-time feedback to the individual, especially with respect to calorie burn rate. In fact, existing wearables or applications are not able to accurately identify the current activity being performed by a given user. In contrast, the current invention is accurately able to identify the activity being performed by the user. It may be notes that identification of the activity is pivotal to arrive at an accurate estimate of calories burned by the user while and after performing the activity.

In light of the above-mentioned advantages and the technical advancements provided by the disclosed method and system, the claimed steps as discussed above are not routine, conventional, or well understood in the art, as the claimed steps enable the following solutions to the existing problems in conventional technologies. Further, the claimed steps clearly bring an improvement in the functioning of the device itself as the claimed steps provide a technical solution to a technical problem.

The specification has described method and system for automated test case generation. The illustrated steps are set out to explain the exemplary embodiments shown, and it should be anticipated that ongoing technological development will change the manner in which particular functions are performed. These examples are presented herein for purposes of illustration, and not limitation. Further, the boundaries of the functional building blocks have been arbitrarily defined herein for the convenience of the description. Alternative boundaries can be defined so long as the specified functions and relationships thereof are appropriately performed. Alternatives (including equivalents, extensions, variations, deviations, etc., of those described herein) will be apparent to persons skilled in the relevant art(s) based on the teachings contained herein. Such alternatives fall within the scope and spirit of the disclosed embodiments.

Furthermore, one or more computer-readable storage media may be utilized in implementing embodiments consistent with the present disclosure. A computer-readable storage medium refers to any type of physical memory on which information or data readable by a processor may be stored. Thus, a computer-readable storage medium may store instructions for execution by one or more processors, including instructions for causing the processor(s) to perform steps or stages consistent with the embodiments described herein. The term “computer-readable medium” should be understood to include tangible items and exclude carrier waves and transient signals, i.e., be non-transitory. Examples include random access memory (RAM), read-only memory (ROM), volatile memory, nonvolatile memory, hard drives, CD ROMs, DVDs, flash drives, disks, and any other known physical storage media.

It is intended that the disclosure and examples be considered as exemplary only, with a true scope and spirit of disclosed embodiments being indicated by the following claims.

What is claimed is:

1. A method for calculating calories burned during activity performance using Artificial Intelligence (AI), the method comprising:
receiving, in real-time, a video stream of a user performing an activity and a set of user profile attributes of the user, wherein the video stream comprises a plurality of frames;
creating, for each of the plurality of frames, a multimedia vector corresponding to the associated frame;
processing, by an AI model, the multimedia vector created for each of the plurality of frames to determine a set of activity parameters associated with the user;
selecting, by the AI model, a target user from a plurality of target users based on similarity between the set of user profile attributes of the user and a set of target profile attributes of the target user, wherein the target user corresponds to a benchmark user;
identifying a set of target activity parameters associated with the target user;
comparing, by the AI model, each of the set of activity parameters with a corresponding activity parameter from the set of target activity parameters;
determining in real-time, contemporaneous to the user performing the activity, a user efficiency level based on a result of the comparing; and
calculating, by the AI model, in real-time, contemporaneous to the user performing the activity, a count of calories burned by the user based on the user efficiency level.

2. The method of claim 1, wherein the set of activity parameters comprises a type of activity, a pace of performing the activity, number of repetitions of the activity, periodicity of the activity, duration of performing the activity, an accuracy of performing the activity, and a degree of freedom of the activity.

3. The method of claim 1, wherein the set of profile attributes comprises age, gender, height, weight, and physical activities.

4. The method of claim 1, wherein identifying the set of target activity parameters further comprises:
receiving a video of the target user performing a target activity and the set of target profile attributes of the target user, wherein the video comprises a plurality of frames;
creating, for each of the plurality of frames, a multimedia vector corresponding to the associated frame; and
processing, by the AI model, the multimedia vector created for each of the plurality of frames to determine the set of target activity parameters associated with the target user.

5. The method of claim 4, further comprising:
determining, by the AI model, an estimated target calories for the target activity based on a count of calories burned by the target user corresponding to the target activity; and
setting a target efficiency level corresponding to the target activity for the target user.

6. The method of claim 4, further comprising:
storing the set of target activity parameters associated with the target user in a database.
storing the target efficiency level within the database.

7. The method of claim 1, wherein selecting the target user from the plurality of target users further comprises:
determining, by the AI model, similarity of the set of user profile attributes with the corresponding set of target profile attributes of each of the plurality of target users, wherein determining the similarity comprises:
calculating, by the AI model, a similarity score between each of the set of user profile attributes and each of the corresponding set of target profile attributes; and
selecting, by the AI model, the target user based on the calculated similarity score, wherein the similarity score calculated for the target user is the highest.

8. The method of claim 1, further comprising:
rendering, by the AI model via a Graphical User Interface (GUI), efficiency level of the user, the count of calories burned by the user, the set of user activity parameters, the set of target activity parameters on a user device.

9. A system for calculating calories burned during activity performance using AI, the system comprising:
a processor; and
a memory communicatively coupled to the processor, wherein the memory stores processor executable instructions, which, on execution, causes the processor to:
receive, in real-time, a video stream of a user performing an activity and a set of user profile attributes of the user, wherein the video stream comprises a plurality of frames;
create, for each of the plurality of frames, a multimedia vector corresponding to the associated frame;
process, by an AI model, the multimedia vector created for each of the plurality of frames to determine a set of activity parameters associated with the user;
select, by the AI model, a target user from a plurality of target users based on similarity between the set of user profile attributes of the user and a set of target profile attributes of the target user, wherein the target user corresponds to a benchmark user;
identify a set of target activity parameters associated with the target user;
compare, by the AI model, each of the set of activity parameters with a corresponding activity parameter from the set of target activity parameters;
determine in real-time, contemporaneous to the user performing the activity, a user efficiency level based on a result of the comparing; and
calculate, by the AI model, in real-time, contemporaneous to the user performing the activity, a count of calories burned by the user based on the user efficiency level.

10. A method for calculating calories burned during activity performance using Artificial Intelligence (AI), the method comprising:
receiving, in real-time, a video stream of a user performing an activity and a set of user profile attributes of the user, wherein the video stream comprises a plurality of frames;
creating, for each of the plurality of frames, a multimedia vector corresponding to the associated frame;
processing, by an AI model, the multimedia vector created for each of the plurality of frames to determine a set of activity parameters associated with the user;
comparing, by the AI model, each of the set of activity parameters with a corresponding activity parameter from a set of target activity parameters, wherein the set of target activity parameters corresponds to the activity being performed by a benchmark user;
determining in real-time, contemporaneous to the user performing the activity, a user efficiency level based on a result of the comparing;
adapting, by the AI model, the user efficiency level based on the set of user profile attributes of the user and a set of benchmark profile attributes of the benchmark user; and
calculating, by the AI model, in real-time, contemporaneous to the user performing the activity, a count of calories burned by the user based on the adapted user efficiency level.

11. The method of claim 10, wherein the set of activity parameters comprises a type of activity, a pace of performing the activity, number of repetitions of the activity, periodicity of the activity, duration of performing the activity, an accuracy of performing the activity, and a degree of freedom of the activity.

12. The method of claim 10, wherein the set of profile attributes comprises age, gender, height, weight, and physical activities.

13. The method of claim 10, further comprising:
receiving, by the AI model, a video of the benchmark user performing a target activity and the set of the benchmark profile attributes of the benchmark user, wherein the video comprises a plurality of frames;
creating, for each of the plurality of frames, a multimedia vector corresponding to the associated frame; and
processing, by the AI model, the multimedia vector created for each of the plurality of frames to determine the set of target activity parameters associated with the benchmark user.

14. The method of claim 13, further comprising:
determining, by the AI model, an estimated target calories for the target activity based on a count of calories burned by the benchmark user corresponding to the target activity; and
setting a benchmark efficiency level corresponding to the target activity.

15. The method of claim 13, further comprising:
storing the set of target activity parameters associated with the benchmark user in a database.
storing the benchmark efficiency level within the database.

16. The method of claim 10, wherein adapting comprises:
computing, by the AI model, a deviation of each of the set of user profile attributes relative to the associated attributes from the set of benchmark profile attributes; and
adjusting, by the AI model, the user efficiency level based on the computed deviation for each of the set of user profile attributes.

17. The method of claim 10, further comprising:
rendering, by the AI model via a Graphical User Interface (GUI), efficiency level of the user, the count of calories burned by the user, the set of user activity parameters, the set of target activity parameters on a user device.

18. A system for calculating calories burned during activity performance using AI, the system comprising:
a processor; and
a memory communicatively coupled to the processor, wherein the memory stores processor executable instructions, which, on execution, causes the processor to:
receive, in real-time, a video stream of a user performing an activity and a set of user profile attributes of the user, wherein the video stream comprises a plurality of frames;
create, for each of the plurality of frames, a multimedia vector corresponding to the associated frame;
process, by an AI model, the multimedia vector created for each of the plurality of frames to determine a set of activity parameters associated with the user;
compare, by the AI model, each of the set of activity parameters with a corresponding activity parameter from a set of target activity parameters, wherein the set of target activity parameters corresponds to the activity being performed by a benchmark user;
determine, in real-time, contemporaneous to the user performing the activity, a user efficiency level based on a result of the comparing;
adapt, by the AI model, the user efficiency level based on the set of user profile attributes of the user and a set of benchmark profile attributes of the benchmark user; and
calculate in real-time, contemporaneous to the user performing the activity, a count of calories burned by the user based on the adapted user efficiency level.
